(19) United States
(12) Patent Application Publication
(10) Pub. No.: US 2024/0374545 A1
(43) Pub. Date: Nov. 14, 2024

(54) DIAGNOSIS OF IMMUNITY REDUCTION THROUGH INTESTINAL MICROFLORA ANALYSIS OF STOMACH CANCER PATIENTS, AND THERAGNOSTIC COMPOSITION USING INTESTINAL MICROFLORA

(71) Applicant: THE CATHOLIC UNIVERSITY OF KOREA INDUSTRY-ACADEMIC COOPERATION FOUNDATION, Seoul (KR)

(72) Inventors: Kyo Young SONG, Seoul (KR); Mi La CHO, Seoul (KR); Joo Yeon JHUN, Seoul (KR); Yoon Ju JUNG, Seoul (KR); Sojung KIM, Seoul (KR); Seung Yoon LEE, Seoul (KR); Jin Seok WOO, Uijeongbu-si (KR); Kun Hee LEE, Seoul (KR)

(73) Assignee: THE CATHOLIC UNIVERSITY OF KOREA INDUSTRY-ACADEMIC COOPERATION FOUNDATION, Seoul (KR)

(21) Appl. No.: 18/778,440

(22) Filed: Jul. 19, 2024

Related U.S. Application Data
(63) Continuation of application No. PCT/KR2023/001123, filed on Jan. 25, 2023.

(30) Foreign Application Priority Data
Jan. 21, 2022 (KR) 10-2022-0009014
Jan. 21, 2022 (KR) 10-2022-0009015
Jan. 25, 2023 (KR) 10-2023-0009490
Jan. 25, 2023 (KR) 10-2023-0009492

(51) Int. Cl. A61K 31/155; A61K 35/74; A61P 35/00; A61P 37/04

(52) U.S. Cl. CPC A61K 31/155; A61K 35/74; A61P 35/00; A61P 37/04

(57) ABSTRACT

The method for preventing or treating of cancer includes administering a pharmaceutical composition to a subject in need thereof comprising a compound represented by Chemical Formula 1 below or a pharmaceutically acceptable salt thereof as an active ingredient.

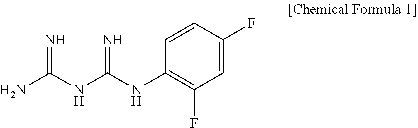 [SMILES: N=C(N)NC(=N)Nc1ccc(F)cc1F]

wherein the composition inhibits the growth of cancer cells.

### CROSS-REFERENCE TO RELATED APPLICATION

Pursuant to 35 USC 120 and 365(c), this application is a continuation of International Application No. PCT/KR2023/001123 filed on Jan. 25, 2023, and claims the benefit under 35 USC 119(a) of Korean Application No. 10-2022-0009014 filed on Jan. 21, 2022, Korean Application No. 10-2022-0009015 filed on Jan. 21, 2022, Korean Application No. 10-2023-0009490 filed on Jan. 25, 2023, and Korean Application No. 10-2023-0009492 filed on Jan. 25, 2023, in the Korean Intellectual Property Office, the entire disclosures of which are incorporated herein by reference for all purposes.

### TECHNICAL FIELD

The present invention relates to the diagnosis of immunity reduction through intestinal microflora analysis of stomach cancer patients, and a theragnostic composition using intestinal microflora.

### BACKGROUND ART

In order to treat cancer, researchers around the world are conducting numerous studies on various topics about intracellular signaling mechanisms and metastasis of cancer cells and side effects of drugs, and every year, novel drugs have been developed and clinical trials have been conducted to be expected to have therapeutic effects on many cancer patients. In addition to removal of tumor tissue through surgery and radiation therapy, current anticancer agents used in anticancer therapy are applied differently depending on each cancer type. In general, chemotherapy agents prescribed to cancer patients target cancer cells, and have the effect of killing cancer cells, but also affect normal cells, thereby causing side effects, such as hair loss, fever, reduced immunity, etc. in patients. Thereafter, based on genetic research on cancer and the like, targeted anticancer agents targeting genetic mutations occurring in each cancer type were developed to improve side effects caused by conventional chemotherapy agents, but there is a problem in that cancer cells, which adapt very quickly to the environment, cause anticancer agent resistance to escape the attack of targeted anticancer agents, so that 100% continuous cancer therapy effects by targeted anticancer agents may not be expected. Recently, research on anticancer agents and the tumor microenvironment has been actively conducted, and cancer immunotherapy agents against various immune checkpoint inhibitors that regulate the immunity of patients while maintaining the anticancer effects have been developed and used as therapeutic agents for patients. Among them, treatment against PD-1/PD-L1 is known to show a high therapeutic response in patients with skin cancer, lung cancer, etc. These cancer immunotherapy agents have the function of inhibiting the proliferation of cancer cells and increasing the activity of immune cells by regulating the function of immune cells within the tumor microenvironment. However, the cancer immunotherapy agents do not show the same anticancer effect in all patients, and biomarkers for cancer immunotherapy agents have not been clearly identified, and there are problems that anticancer agent resistance occurs due to JAK-STAT genetic mutation, autoimmune diseases may be caused due to the characteristics of antibody compounds, and expensive treatment costs occur.

Meanwhile, a microbiome refers to microorganisms that live in the human body and corresponds to intestinal microorganisms. The number of microbiomes is at least twice greater than that of pure human cells, and the number of genes is at least 100 times greater therethan. Accordingly, since it is impossible to discuss human genes without mentioning microorganisms, the microbiome is also called a second genome. The microbiome is a field that may be widely used in the development of new drugs and research on treatments for incurable diseases by analyzing the principles of formation of beneficial and harmful bacteria, association between diseases, etc. In addition, the microbiomes are used to develop foods, cosmetics, and treatments.

As next generation sequencing (NGS) technology advances, the importance of intestinal microorganisms is emerging in human physiology and immune regulation, and thus the importance of probiotics capable of directly regulating the structure of a human intestinal microbiome is emerging. Recently, through many studies, as probiotics are known to be closely related to the human microbiome, the probiotics have attracted attention for their functions as regulators that change an intestinal environment. The intake of probiotics is known to not only promote digestion through the microbiome regulation function, but also suppress inflammatory bowel disease, infectious diseases, and harmful bacteria. In particular, it has been found that the probiotics improve the host immune system to have an effect on various immune-related diseases including atopy and rheumatism and cancers.

In addition, as the probiotics field grows significantly, much attention has also focused on prebiotics, synbiotics, and postbiotics. The prebiotics are defined as ‘substances that are selectively used by beneficial bacteria that contribute to health among the microorganisms in the host’, and representative examples thereof include dietary fiber and oligosaccharides that serve as food for lactic acid bacteria. The synbiotics are a form that contains both probiotics and prebiotics. Meanwhile, the postbiotics, which have recently been attracting attention, are materials that contain useful metabolites produced by probiotics and components of microorganisms and are clearly defined as ‘a non-living form of microorganisms beneficial to the health of the host or a formulation containing components of those microorganisms.’ The postbiotics are attracting attention as a new alternative material that may overcome the limitations of safety, functionality, and stability of conventional probiotic materials.

Therefore, the present inventors confirmed that a novel compound may inhibit the expression of PD-L1, a cancer cell surface protein, and effectively kill cancer cells to inhibit the evasion of immune responses of cancer cells and increase the activity of immune cells. In addition, the present inventors identified and selected microbiomes with reduced community diversity and taxa in cancer patient groups, confirmed that the selected strains improved the immunity of cancer patient groups, and the metabolites of the strains also improved immune responses to have excellent effects as cancer immunotherapy agents, and then completed the present invention.

### DISCLOSURE

### Technical Problem

An object of the present invention is to provide a pharmaceutical composition for the prevention or treatment of cancer including a compound represented by Chemical Formula 1 below or a pharmaceutically acceptable salt thereof as an active ingredient.

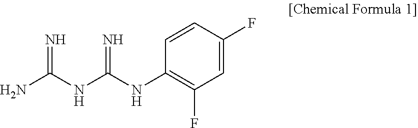 [SMILES: N=C(N)NC(=N)Nc1ccc(F)cc1F]

Another object of the present invention is to provide an anticancer adjuvant including the compound represented by Chemical Formula 1 or a pharmaceutically acceptable salt thereof as an active ingredient.

Yet another object of the present invention is to provide a food composition for the prevention or improvement of cancer including the compound represented by Chemical Formula 1 or a pharmaceutically acceptable salt thereof as an active ingredient.

Yet another object of the present invention is to provide a pharmaceutical composition for the prevention or treatment of cancer with improved expression of Programmed Death-Ligand 1 (PD-L1) including the compound represented by Chemical Formula 1 or a pharmaceutically acceptable salt thereof as an active ingredient.

Yet another object of the present invention is to provide a method for treating cancer including administering the compound represented by Chemical Formula 1 or a pharmaceutically acceptable salt thereof in a pharmaceutically effective amount to a subject.

Yet another object of the present invention is to provide a pharmaceutical composition for the prevention or treatment of cancer including butyrate or a microbiome as an active ingredient.

Yet another object of the present invention is to provide a food composition for the prevention or improvement of cancer including butyrate or a microbiome as an active ingredient.

Yet another object of the present invention is to provide a method for increasing Faecalibacterium sp. strains and decreasing Proteobacteria Phylum strains in cancer patient groups, including administering a composition including butyrate or a microbiome as an active ingredient to a subject.

Yet another object of the present invention is to provide a method for increasing a T cell function and an antigen presenting cell (APC) function in cancer patient groups, including administering a composition including butyrate or a microbiome as an active ingredient to a subject.

Yet another object of the present invention is to provide a method for treating cancer, including administering butyrate or a microbiome in a pharmaceutically effective amount to a subject.

### Technical Solution

One aspect of the present invention provides a pharmaceutical composition for the prevention or treatment of cancer including a compound represented by Chemical Formula 1 below or a pharmaceutically acceptable salt thereof as an active ingredient.

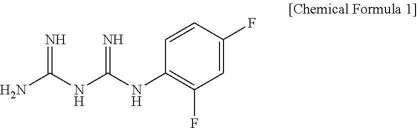 [SMILES: N=C(N)NC(=N)Nc1ccc(F)cc1F]

In addition, another aspect of the present invention provides an anticancer adjuvant including the compound represented by Chemical Formula 1 or a pharmaceutically acceptable salt thereof as an active ingredient.

In addition, yet another aspect of the present invention provides a food composition for the prevention or improvement of cancer including the compound represented by Chemical Formula 1 or a pharmaceutically acceptable salt thereof as an active ingredient.

In addition, yet another aspect of the present invention provides a pharmaceutical composition for the prevention or treatment of cancer with improved expression of Programmed Death-Ligand 1 (PD-L1) including the compound represented by Chemical Formula 1 or a pharmaceutically acceptable salt thereof as an active ingredient.

In addition, yet another aspect of the present invention provides a method for treating cancer including administering the compound represented by Chemical Formula 1 or a pharmaceutically acceptable salt thereof in a pharmaceutically effective amount to a subject.

In addition, yet another aspect of the present invention provides a pharmaceutical composition for the prevention or treatment of cancer including butyrate or a microbiome as an active ingredient.

In addition, yet another aspect of the present invention provides a food composition for the prevention or improvement of cancer including butyrate or a microbiome as an active ingredient.

In addition, yet another aspect of the present invention provides a method for increasing Faecalibacterium sp. strains and decreasing Proteobacteria Phylum strains in cancer patient groups, including administering a composition including butyrate or a microbiome as an active ingredient to a subject.

In addition, yet another aspect of the present invention provides a method for increasing a T cell function and an antigen presenting cell (APC) function in cancer patient groups, including administering a composition including butyrate or a microbiome as an active ingredient to a subject.

In addition, yet another aspect of the present invention provides a method for treating cancer, including administering butyrate or a microbiome in a pharmaceutically effective amount to a subject.

### Advantageous Effects

According to the present invention, it has been identified that a novel compound SD217 of the present invention specifically kills stomach cancer cells and effectively inhibits the expression of PD-L1, which is a surface protein expressed by stomach cancer cells in order to evade immune responses of the human body, and thus kills stomach cancer cells and enhances the immune function. In addition, it has been identified that intestinal microflora diversity decreases in stomach cancer patients and the composition ratio of the intestinal microflora differs from those of healthy adults and benign tumor patients. In addition, it has been identified that the immune function declines in stomach cancer patient groups and the immune function of patients declines as the stomach cancer progresses. In addition, it has been identified that PD-L1 and IL-10, which increase because of reduced immunity, are effectively inhibited when macrophages derived from patients are treated with a Faecalibacterium prausnitzii strain, which has been identified to be reduced in stomach cancer patient groups, and butyrate, which is a metabolite thereof. In addition, it has been identified that cell killing is effectively induced when a stomach cancer cell line is treated with a Faecalibacterium prausnitzii strain and butyrate, which is a metabolite thereof. In addition, it has been identified that even in a stomach cancer avatar animal model in which the immune status of patients is reflected and into which a stomach cancer cell line is transplanted, butyrate inhibits tumor growth and reduces the expression of tumor markers and immunity reduction markers. Therefore, intestinal microflora dysbiosis and reduced immunity in stomach cancer patient groups are diagnosed such that the effects of increased immunity and intestinal microflora recovery according to the treatment of an active ingredient of the present invention have been identified, and thus it has been identified that individual personalized medicine may be implemented.

### DESCRIPTION OF DRAWINGS

FIG. 1 is a diagram of confirming apoptosis of a stomach cancer cell line according to treatment with SD217 of the present invention by flow cytometry (A: flow cytometry results, B: quantification of analysis results).

FIG. 2 is a diagram of analyzing the expression of PD-L1, a surface protein of a stomach cancer cell line, according to treatment with SD217 of the present invention by flow cytometry (A: flow cytometry results, B: quantification of analysis results).

FIG. 3 is a diagram of confirming the inhibition of tumor growth by administering SD217 of the present invention to mice with stomach cancer tumor (A: confirmation of tumor growth inhibition, B: quantification of tumor volume).

FIG. 4 is a diagram of quantifying early and late apoptosis of SD217 of the present invention.

FIG. 5 is a diagram of comparing the diversity of intestinal microflora in stomach cancer patients and healthy adults (A: comparison of intestinal microflora diversity, B: comparison of reduced intestinal microflora).

FIG. 6 is a diagram of analyzing the diversity of intestinal microflora in stomach cancer patients and healthy adults using α-diversity and β-diversity (A: Observed OTU and Chaol analysis results, B: PCoA plot and bray curstis distance analysis results).

FIG. 7 is a diagram of comparing composition ratios of intestinal microflora in stomach cancer patients and benign tumor patients (A: comparison of composition ratios of intestinal microflora, B: comparison of Faecalibacterium sp. composition).

FIG. 8 is a diagram of comparing expression of immunity reduction factors of T cells in stomach cancer patients and benign tumor patients (A: confirmed IL-10 expression, B: confirmed IL-17 expression, C: confirmed INF-γ expression).

FIG. 9 is a diagram of comparing expression of PD-1, an immunity reduction factor of T cells in stomach cancer patients and benign tumor patients (A: comparison of CD4+ cells, B: comparison of CD8+ cells).

FIG. 10 is a diagram of comparing expression of PD-L1, an immunity reduction factor of antigen presenting cells in stomach cancer patients and benign tumor patients (A: comparison of CD68+ cells, B: comparison of CD11+ cells).

FIG. 11 is a diagram of comparing expression of IL-10, an immunity reduction factor of antigen presenting cells in stomach cancer patients and benign tumor patients (A: comparison of CD68+ cells, B: comparison of CD11+ cells).

FIG. 12 is a diagram of comparing expression of PD-1 and CTLA-4, immunity reduction factors of T cells in early stomach cancer patients and advanced stomach cancer patients (A: comparison of PD-1 expression, B: comparison of CTLA-4 expression).

FIG. 13 is a diagram of comparing expression of PD-L1 and IL-10, immunity reduction factors of antigen presenting cells in early stomach cancer patients and advanced stomach cancer patients (A: comparison of PD-L1 expression, B: comparison of IL-10 expression).

FIG. 14 is a diagram of comparing expression of PD-L1, an immunity reduction factor of antigen presenting cells according to stomach cancer progression of stomach cancer patients (A: comparison of CD68+ cells, B: comparison of CD11+ cells).

FIG. 15 is a diagram of confirming expression of PD-L1 and IL-10, immunity reduction factors in stomach cancer tissue according to stomach cancer progression of stomach cancer patients using a confocal microscope (A: analysis results, B: quantification of analysis results).

FIG. 16 is a diagram of comparing the expression of PD-L1 and IL-10 according to treatment with butyrate in macrophages of stomach cancer patients (A: comparison of PD-L1 expression, B: comparison of IL-10 expression).

FIG. 17 is a diagram of comparing the expression of PD-L1 according to treatment with Faecalibacterium prausnitzii in macrophages of stomach cancer patients.

FIG. 18 is a diagram of confirming the expression of immunity reduction markers according to treatment with butyrate or Faecalibacterium prausnitzii in macrophages of stomach cancer patients (A: quantification of marker expression in macrophages, B: quantification of marker expression in culture medium, C: quantification of marker expression according to treatment with Faecalibacterium prausnitzii).

FIG. 19 is a diagram of confirming apoptosis by flow cytometry according to treatment with Faecalibacterium prausnitzii of the present invention in a stomach cancer cell line (A: flow cytometry results, B: quantification of analysis results).

FIG. 20 is a diagram of confirming apoptosis by flow cytometry according to treatment with butyrate in a stomach cancer cell line (A: flow cytometry results, B: quantification of analysis results).

FIG. 21 is a diagram of confirming cell viability using an absorbance according to treatment with butyrate in a stomach cancer cell line.

FIG. 22 is a diagram of confirming the inhibition of tumor growth according to treatment with butyrate in an immune-simulation avatar animal model of stomach cancer patients (A: tumor growth inhibition results, B: quantification of tumor volume).

FIG. 23 is a diagram of confirming the expression of tumor markers and immunity reduction markers in tumor tissue according to treatment with butyrate by immunohistochemical staining in an immune-simulation avatar animal model of stomach cancer patients (A: staining results, B: quantification of staining results).

### BEST MODE OF THE INVENTION

The present invention provides a pharmaceutical composition for the prevention or treatment of cancer including a compound represented by Chemical Formula 1 below or a pharmaceutically acceptable salt thereof as an active ingredient.

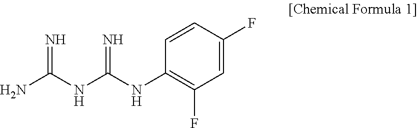 [SMILES: N=C(N)NC(=N)Nc1ccc(F)cc1F]

The term “SD217 compound” of the present invention may be a compound (1-(2,4-difluorophenyl)biguanide) represented by Chemical Formula 1, and a specific preparation method is described in Preparation Example 1 below.

As used in the present invention, the term “prevention” refers to all actions that suppress the symptoms of a specific disease or delay its progression by administering the composition of the present invention.

As used in the present invention, the term “treatment” refers to all actions that improve or beneficially change the symptoms of a specific disease by administering the composition of the present invention.

The pharmaceutically acceptable salt may include an acid addition salt formed by a pharmaceutically acceptable free acid, and the free acid may use organic acids and inorganic acids. The organic acids may include citric acid, acetic acid, lactic acid, tartaric acid, maleic acid, fumaric acid, formic acid, propionic acid, oxalic acid, trifluoroacetic acid, benzoic acid, gluconic acid, metasulfonic acid, glycolic acid, succinic acid, 4-toluenesulfonic acid, glutamic acid, and aspartic acid, but are not limited thereto. In addition, the inorganic acids may include hydrochloric acid, hydrobromic acid, sulfuric acid, phosphoric acid, and the like, but are not limited thereto.

The pharmaceutical composition of the present invention may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present invention may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present invention is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present invention may be formulated and used in the form of oral formulations, such as powders, granules, tablets, capsules, suspensions, emulsions, syrups, aerosols, etc., external preparations, suppositories, and sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one excipient, for example, starch, calcium carbonate, sucrose, lactose, gelatin, etc. with the active ingredients. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base of the suppository, witepsol, Tween 61, cacao butter, laurinum, glycerogelatin, etc. may be used.

The pharmaceutical composition of the present invention may be administered to a subject through various routes. All methods of administration may be expected, and the pharmaceutical composition may be administered, for example, oral, intravenous, intramuscular, subcutaneous, and intraperitoneal injection.

The dose of the pharmaceutical composition according to the present invention is selected in consideration of the age, body weight, sex, and physical conditions of the subject. It is obvious that the concentration of the active ingredients included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be shown, and when the concentration exceeds 5,000 μg/ml, toxicity to the human body may be exhibited.

According to an embodiment of the present invention, the composition may increase apoptosis of cancer cells, and the apoptosis may be early or late apoptosis.

According to an embodiment of the present invention, the composition may inhibit the growth of cancer cells.

According to an embodiment of the present invention, the composition may inhibit the expression of Programmed Death-Ligand 1 (PD-L1), which is a surface protein of cancer cells.

The “PD-L1” and “PD-1” of the present invention are surface proteins of cancer cells or hematopoietic cells and surface proteins of T cells, proteins for inducing cancer cells to evade the immune responses of T cells when the T cell surface proteins PD-1 and PD-L1 are combined, and proteins that PD-L1 is overexpressed in cancer cells and hematopoietic cells including antigen-presenting cells, PD-1 is overexpressed in T cells, and the expression of PD-L1 and PD-1 complex increases, and thus cancer cell specificity of T cells decreases, resulting in declining the immune function.

According to an embodiment of the present invention, the cancer may be cancer selected from the group consisting of stomach cancer, colon cancer, rectal cancer, anal cancer, bone cancer, cerebrospinal tumor, head and neck cancer, thymoma, mesothelioma, esophageal cancer, biliary tract cancer, bladder cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer, preferably stomach cancer, but is not limited thereto.

Further, the present invention provides an anticancer adjuvant including the compound represented by Chemical Formula 1 or a pharmaceutically acceptable salt thereof as an active ingredient.

The “anticancer adjuvant” of the present invention is an agent that may improve, enhance, or increase an anticancer effect of an anticancer agent, and does not show anticancer activity by itself, but may be an agent capable of improving, enhancing, or increasing the anticancer effect of the anticancer agent when used together with the anticancer agent. In addition, when an agent exhibiting a concentration-dependent anticancer activity is used together with the anticancer agent at a level that does not exhibit anticancer activity by itself, the anticancer adjuvant may be an agent capable of improving, enhancing, or increasing the anticancer effect of the anticancer agent.

The anticancer adjuvant may be administered through any general route as long as the anticancer adjuvant may reach a target tissue. The anticancer adjuvant of the present invention may be administered intraperitoneally, intravenously, intramuscularly, subcutaneously, intradermally, orally, intranasally, pulmonaryly, or intrarectally according to a desired purpose, but is not limited thereto. In addition, the anticancer adjuvant may be administered by any device capable of transferring an active substance to a target cell.

Further, the present invention provides a food composition for the prevention or improvement of cancer including the compound represented by Chemical Formula 1 or a pharmaceutically acceptable salt thereof as an active ingredient.

As used in the present invention, the term “improvement” means all actions that at least reduce parameters associated with conditions to be treated, such as the degree of symptoms.

In addition to containing the active ingredient of the present invention, the food composition of the present invention may contain various flavoring agents or natural carbohydrates as an additional ingredient, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present invention may be formulated in the same manner as the pharmaceutical composition to be used as a functional food or added to various foods. The foods capable of adding the composition of the present invention include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present invention may contain pulp for preparing natural fruit juice, fruit juice beverages, and vegetable beverages.

The functional food composition of the present invention may be prepared and processed in the form of tablets, capsules, powders, granules, liquids, pills, etc. for the purpose of prevention or treatment of cancer. In the present invention, the ‘health functional food composition’ refers to foods prepared and processed by using raw materials or ingredients with functionality, which are useful for the human body according to the Art on Health Functional Foods No. 6727, and means foods taken for adjusting nutrients for the structures and functions of the human body or obtaining a useful effect on health applications such as physiological actions. The health functional food of the present invention may include conventional food additives, and the suitability as the food additives is determined by the specifications and standards for the corresponding item in accordance with the general rules of the Food Additive Codex, general test methods, etc., that are approved by the Food and Drug Administration, unless otherwise specified. The items disclosed in the ‘Food Additives Codex’ may include, for example, chemical composites such as ketones, glycine, calcium citrate, nicotinic acid, cinnamic acid, etc.; natural additives such as desensitizing dye, licorice extract, crystal cellulose, Kaoliang color, guar gum, etc.; mixed formulations such as sodium L-glutamic acid formulations, noodle additive alkali agents, preservative formulations, tar color formulations, etc. For example, the health functional food in the form of tablets may be formed by granulating a mixture obtained by mixing the active ingredient of the present invention with an excipient, a binder, a disintegrant, and other additives by conventional methods, and then compression-molding the mixture by adding a slip modifier and the like, or directly compressing the mixture. In addition, the health functional food in the form of tablets may also contain a flavors enhancer or the like as needed. In the health functional food in the form of capsules, hard capsules may be prepared by filling a mixture mixed with the active ingredient of the present invention and additives such as excipients into conventional hard capsules, and soft capsules may be prepared by filling a mixture mixed with the active ingredient of the present invention and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture mixed with the active ingredient of the present invention and an excipient, a binder, a disintegrant, etc. by conventional known methods, and may also be coated with white sugar or other coating agents or surface-coated with materials such as starch and talc, if necessary. The health functional food in the form of granules may be prepared by granulizing a mixture mixed with the active ingredient of the present invention and an excipient, a binder, a disintegrant, etc. by conventional known methods and may contain a flavoring agent, a flavors enhancer, etc., if necessary.

Further, the present invention provides a pharmaceutical composition for the prevention or treatment of cancer with improved expression of Programmed Death-Ligand 1 (PD-L1), including the compound represented by Chemical Formula 1 or a pharmaceutically acceptable salt thereof as an active ingredient.

According to one embodiment of the present invention, the composition may decrease the expression of PD-L1, and the decreasing of the expression of PD-L1 may increase the cancer-specific activity of immune cells.

Further, the present invention provides a method for treating cancer including administering the compound represented by Chemical Formula 1 or a pharmaceutically acceptable salt thereof in a pharmaceutically effective amount to a subject.

The treatment method of the present invention includes administering the compound represented by Chemical Formula 1 or a pharmaceutically acceptable salt thereof in a therapeutically effective amount to a subject. It is preferred that a specific therapeutically effective amount for a specific subject is differently applied depending on various factors including the kind and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. A daily dose may be 0.0001 to 100 mg/kg, preferably 0.01 to 100 mg/kg, based on the amount of the pharmaceutical composition of the present invention, and may be administered 1 to 6 times a day. However, it is obvious to those skilled in the art that the dosage or dose of each active ingredient does not cause side effects by including the content of each active ingredient too high. Therefore, the effective amount of the composition suitable for the purpose of the present invention is preferably determined in consideration of the aforementioned matters.

The subject is applicable to any mammal, and the mammal includes not only humans and primates, but also livestock such as cattle, pig, sheep, horse, dog, and cat.

A recombinant peptide or recombinant vector of the present invention may be administered to mammals such as mice, rats, livestock, and humans through various routes. All methods of administration may be expected, and for example, the pharmaceutical composition may be administered by oral, rectal or intravenous, intramuscular, subcutaneous, intrauterine dural or intracerebroventricular injection.

The present invention provides a pharmaceutical composition for the prevention or treatment of cancer including butyrate or a microbiome as an active ingredient.

As used in the present invention, the term “microbiome” refers to the entire genetic information of microorganisms inhabiting the human body or the microorganisms themselves, and is a compound word of microbiota, which are microorganisms that inhabit and coexist in the human body, and genome. It is known that the number of human microbiomes is at least twice greater than the number of pure human cells and the number of genes is at least 100 times greater therethan, and the microbiome is a field that may be widely used in research on the microbial environment within the human body, such as development of new drugs and treatment of incurable diseases by analyzing the principles of formation of beneficial and harmful bacteria and the relation between diseases.

According to an embodiment of the present invention, the microbiome may be Faecalibacterium prausnitzii.

According to an embodiment of the present invention, the composition may increase the immune function or may inhibit the expression of IL-10, IL-17, and INF-γ, which are immunity reduction factors.

According to an embodiment of the present invention, the increasing of the immune function may be inhibiting the expression of programmed cell death-1 (PD-1) or CTLA-4 of T cells.

According to an embodiment of the present invention, the increasing of the immune function may be inhibiting the expression of Programmed Death-Ligand 1 (PD-L1) and IL-10 in antigen presenting cells (APC) or cancer tissue, and the antigen presenting cells may be macrophages or dendritic cells.

According to an embodiment of the present invention, the composition may increase apoptosis of cancer cells.

According to an embodiment of the present invention, the cancer may be cancer selected from the group consisting of stomach cancer, colon cancer, rectal cancer, anal cancer, bone cancer, cerebrospinal tumor, head and neck cancer, thymoma, mesothelioma, esophageal cancer, biliary tract cancer, bladder cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer, preferably stomach cancer, but is not limited thereto.

Further, the present invention provides a food composition for the prevention or improvement of cancer including butyrate or a microbiome as an active ingredient. Further, the present invention provides a method for increasing Faecalibacterium sp. strains and decreasing Proteobacteria Phylum strains in cancer patient groups, including administering a composition including butyrate or a microbiome as an active ingredient to a subject.

Further, the present invention provides a method for increasing the T cell function and the Antigen Presenting Cell (APC) function in cancer patient groups, including administering a composition including butyrate or a microbiome as an active ingredient to a subject.

Further, the present invention provides a method for treating cancer, including administering butyrate or a microbiome in a pharmaceutically effective amount to a subject.

### MODES OF THE INVENTION

Hereinafter, the present invention will be described in more detail through Examples. These Examples are to explain the present invention in more detail, and it will be apparent to those skilled in the art that the scope of the present invention is not limited to these Examples.

### <Preparation Example 1> Synthesis and Preparation of SD217

In order to prepare SD217, a novel compound of the present invention, aniline was dissolved in acetonitrile and then added with 1 equivalent of thick hydrochloric acid. After adding dicyano diamide, the reactant was heated to 175° C., and a crystalline material was filtered.

The chemical structure of the material filtered in the crystalline state was confirmed and represented by Chemical Formula 1, which was named 1-(2,4-difluorophenyl) biguanide (SD217).

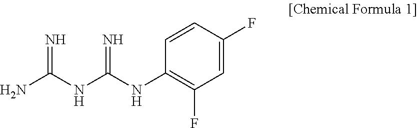 [SMILES: N=C(N)NC(=N)Nc1ccc(F)cc1F]

### <Example 1> Confirmation of Increased Apoptosis of SD217 in Stomach Cancer Cell Line

It was determined whether SD217, a novel compound of the present invention, may specifically kill stomach cancer cells. Specifically, 2×10⁵ of a AGS cell line, a stomach cancer cell line, was inoculated into a 6-well plate and then treated with SD217, the novel compound of the present invention, at a concentration of 0.2 mM after overnight, and after 48 hours, stained with Annexin/PI, and then apoptotic cells were analyzed by flow cytometry. As a control group, a Vehicle group treated with the same amount of PBS was used. Thereafter, early apoptotic cells were confirmed, a ratio of late apoptotic/necrotic cells was confirmed, and thus death of the stomach cancer cell line was confirmed.

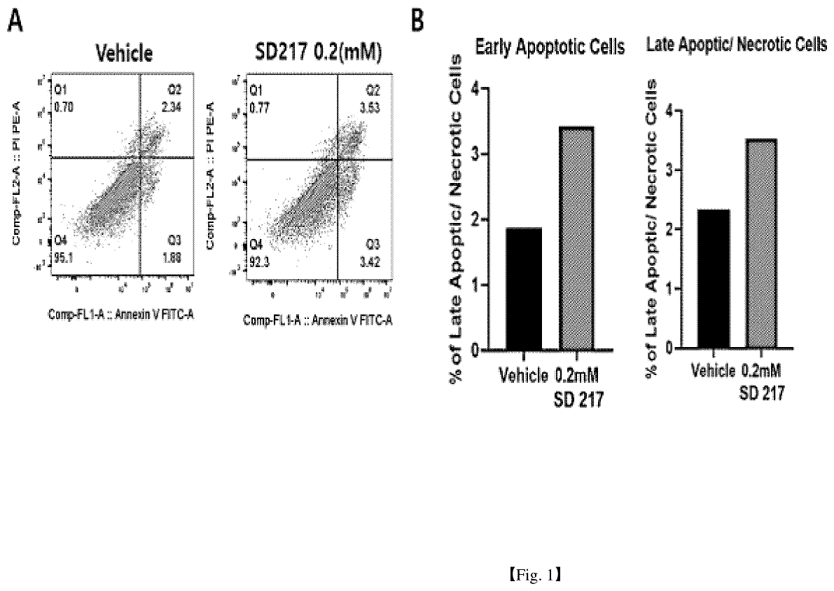

As a result, as shown in FIG. 1, it was confirmed that apoptosis was significantly increased in a group treated with SD217 of the present invention compared to the Vehicle group, and thus it was confirmed that SD217 of the present invention specifically killed stomach cancer cells.

### <Example 2> Confirmation of the Expression of Immunity Reduction Marker of Stomach Cancer Cell by SD217 in Stomach Cancer Cell Line

It was intended to confirm the expression of a Programmed Death-Ligand 1 (PD-L1) protein, an immunity reduction marker of stomach cancer cells, by SD217, a novel compound of the present invention. The PD-L1 protein was a surface protein of stomach cancer cells, and the PD-L1 is a protein that evaded the immune responses of T cells by binding to programmed cell death-1 (PD-1), a surface protein of activated T cells. Further, it was confirmed whether SD217 of the present invention inhibited the expression of PD-L1. Specifically, in the same manner as in Example 1, AGS cells, a stomach cancer cell line, were treated with 0.2, 0.5, and 1 mM of SD217 and reacted. Thereafter, PD-L1 positive cells were analyzed by flow cytometry. As a control group, a Vehicle group treated with PBS was used, and as a positive control group, 1 mM Metformin was used.

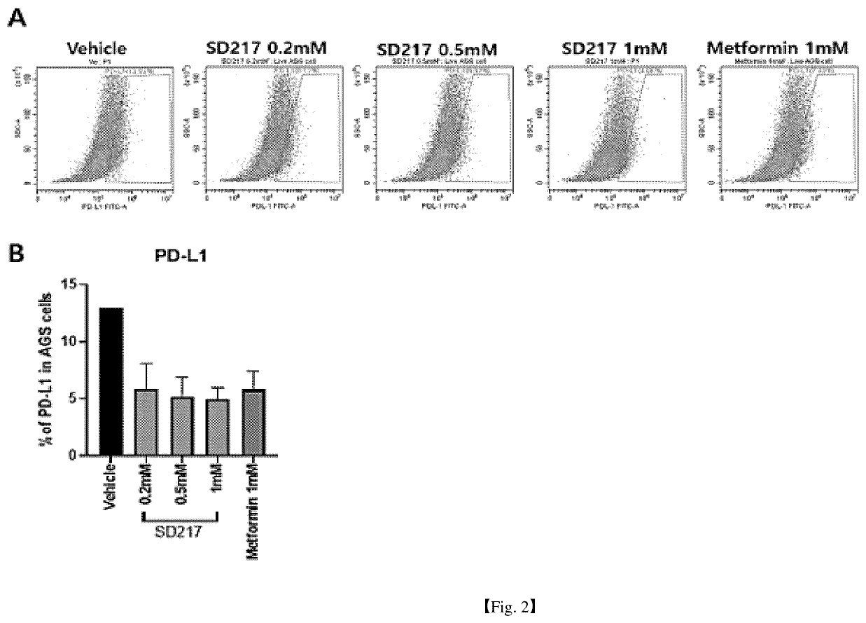

As a result, as shown in FIG. 2, it was confirmed that in stomach cancer cells treated with the SD217 compound of the present invention, the number of PD-L1 positive cells was significantly reduced compared to the Vehicle group.

### <Example 3> Confirmation of Inhibition of Stomach Cancer Tumor Growth

It was confirmed whether SD217, the novel compound of the present invention, inhibited tumor growth. Specifically, a stomach cancer transplant animal model was fabricated by subcutaneously injecting 5×10⁶ of the AGS cell line, a stomach cancer cell line, into 6-8-week-old immunodeficient mice (NSG). Thereafter, SD217 was orally administered daily at a concentration of 50 mg/kg. After AGS cell injection and SD217 administration, tumor size was measured three times weekly using calipers, and tumor volume was calculated by substituting the lengths in long axis and short axis of the tumor into Equation 1 below. As a control group, a Vehicle group injected with the same amount of PBS was used.

$$Tumor volume = 0.52 \times long axis \times short {axis}^{2} \qquad [ Equation 1 ]$$

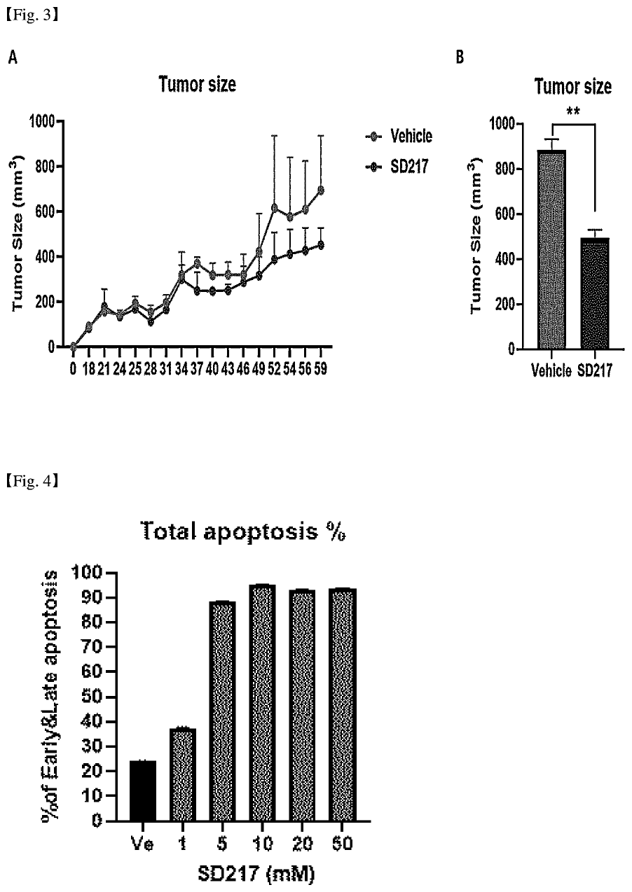

As a result, as shown in FIG. 3, it was confirmed that in the group administered with SD217, compared to the Vehicle group, tumor growth was significantly inhibited, and the tumor volume was significantly reduced, so that SD217 inhibited the tumor growth.

### <Example 4> Confirmation of Apoptosis Induction in Stomach Cancer Cell Line (Early-Late Apoptosis)

It was confirmed whether SD217, the novel compound of the present invention, induced early and late apoptosis in stomach cancer cell line. Specifically, 2×10⁵ of a AGS cell line, a stomach cancer cell line, was inoculated into a 6-well plate and then treated with SD217, the novel compound of the present invention, at concentrations of 1, 5, 10, 20, and 50 mM after overnight, and after 48 hours, stained with Annexin/PI, and then apoptotic cells were analyzed by flow cytometry.

As a result, as shown in FIG. 4, it was confirmed that compared to the Vehicle group, in the group treated with SD217 of the present invention, early and late apoptosis was induced in a concentration-dependent manner.

### <Example 5> Comparison of Intestinal Microflora in Stomach Cancer Patients and Healthy Adults

### <5-1> Comparison of Intestinal Microflora Diversity (Comparison of Stomach Cancer Patient and Healthy Control)

By comparing a stomach cancer (or gastric cancer (GC)) patient with a healthy control (HC) and comparing a stomach cancer (GC) patient with a benign tumor (BT) patient, whether there was a change in intestinal microflora diversity was confirmed. Specifically, the intestinal microflora was compared between healthy adults and stomach cancer patients, and between benign tumor patients and stomach cancer patients. Fecal was collected from the normal control, the benign tumor patient, and the stomach cancer patient and stored at −70° C. until analysis. For metagenomic analysis, bacterial genomic DNA isolation, microorganism-specific 16S rRNA gene amplification, next generation sequencing (NGS), intestinal microflora analysis, and profiling were used. The gDNA was isolated by amplifying genes with primers fabricated from regions that may include V3 and V4 parts of a variant region in a bacterial-specific 16S rRNA gene sequence, attaching an index sequence capable of distinguishing samples and perform twice PCR to create a library, and then decoding a nucleotide sequence using Illumina's Miseq. Intestinal microflora profiling was performed on the decoded sequences by bioinformatic analysis tools, Quantitative Insights into Microbioal Ecology 2 (QIIME 2) and Lefse, and statistical processing using Prism and R. Thereafter, in order to confirm the abundance of the microflora in each group, α-diversity analysis and β-diversity analysis for similarity analysis for each group were performed. In α-diversity analysis, Observed OTUs and Chaol analysis was performed, and in β-diversity analysis, PCoA plot analysis was performed based on bray curstis distance. Thereafter, the composition ratios of the phylum, family, and genus of each group were confirmed.

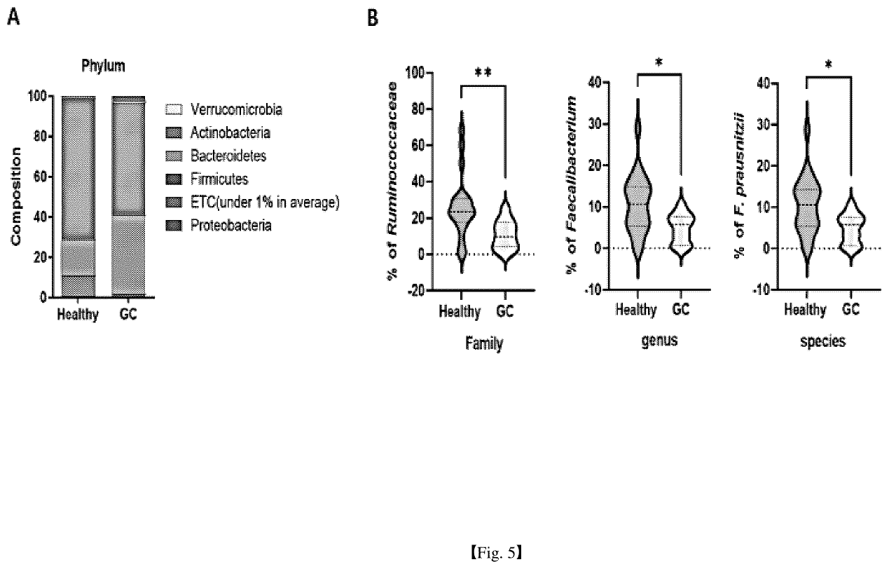

As a result, it was confirmed that in a stomach cancer patient group (GC), compared to a healthy adult (HC), the Proteobacteria phylum was increased and the Actinobacteria phylum was decreased in the intestinal microflora, and in the stomach cancer patient group, the Faecalibacterium species and higher family were significantly reduced (FIG. 5).

In addition, in a-diversity analysis, it was confirmed that Observed OTUs levels were significantly different between the GC group and the H group, and in

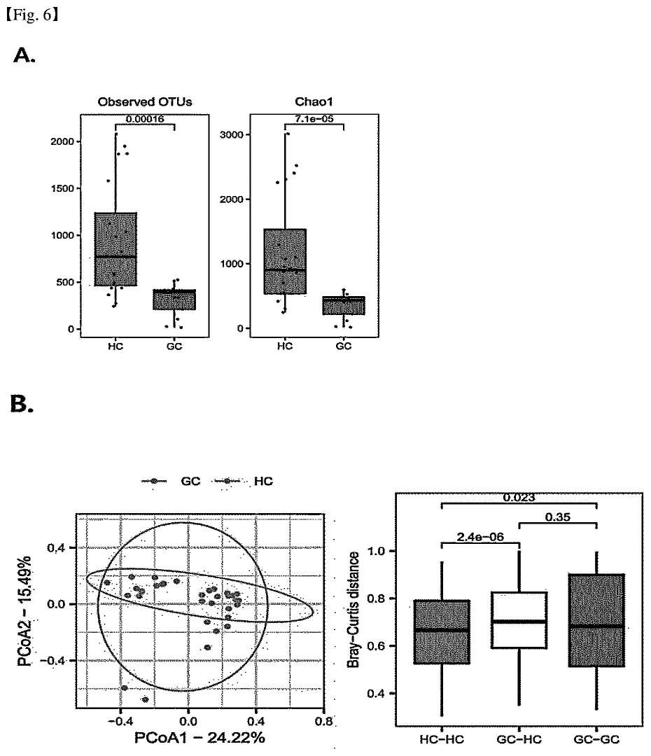

Chaol analysis, it was confirmed that Chaol levels in the GC group were decreased (FIG. 6A). In B-diversity analysis, it was confirmed that the HC group and the GC group were significantly different in permutational multivariate analysis of variance (PERMANOVA), and it was confirmed that a difference between HC and GC significantly increased compared to the difference in each group between HC and GC during bray curstis distance distribution (FIG. 6B).

<5-2> Comparison of Intestinal Microflora Composition (Comparison between Stomach Cancer Patients and Benign Tumor Patients)

It was confirmed whether there was a difference in composition of intestinal microflora between stomach cancer patients and benign tumor patients. In the same manner as in Example 5-1, a difference in composition of the intestinal microflora between a stomach cancer patient group (GC) and a benign tumor patient group (BT) was analyzed up to the Phylum and Genus levels.

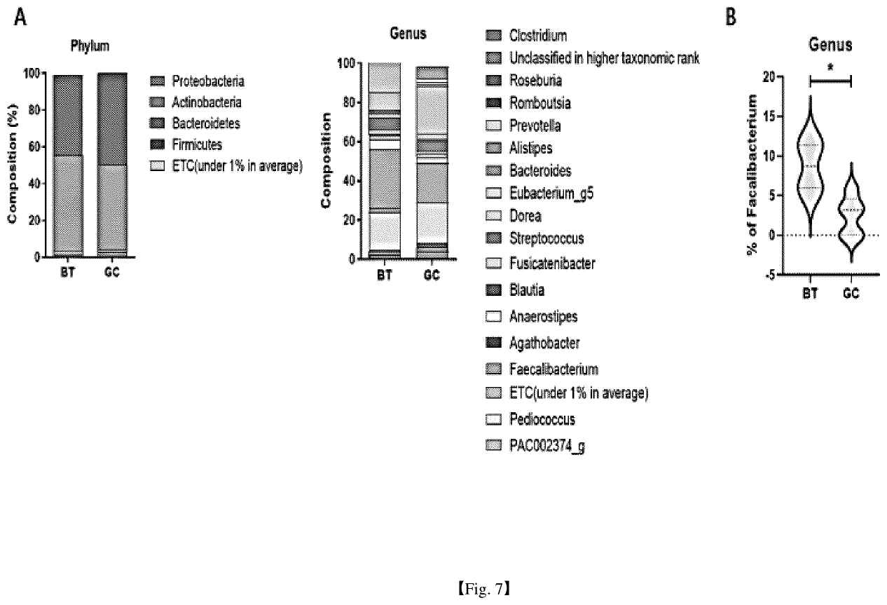

As a result, as shown in FIG. 7, it was further confirmed that in the stomach cancer patient group (GC), compared to the benign tumor patient group (BT), among the intestinal microflora, the Proteobacteria phylum was increased, the Actinobacteriaphylum was reduced, and it was confirmed that the Faecalibacterium genus was significantly reduced. Based on the results of Examples 5-1 and 5-2, it was confirmed that the intestinal microflora became dysbiosis in stomach cancer patients.

### <Example 6> Comparison of Immunity between Stomach Cancer Patients and Benign Tumor Patients

### <6-1> Confirmation of Immunity Reduction of T Cells

In stomach cancer patients and benign tumor patients, whether there was a difference in the immunity of T cells was compared. Specifically, blood was obtained from the stomach cancer patients and benign tumor patients, and the expression of IL-10, IL-17, and IFN-γ related to immunity reduction was analyzed by ELISA.

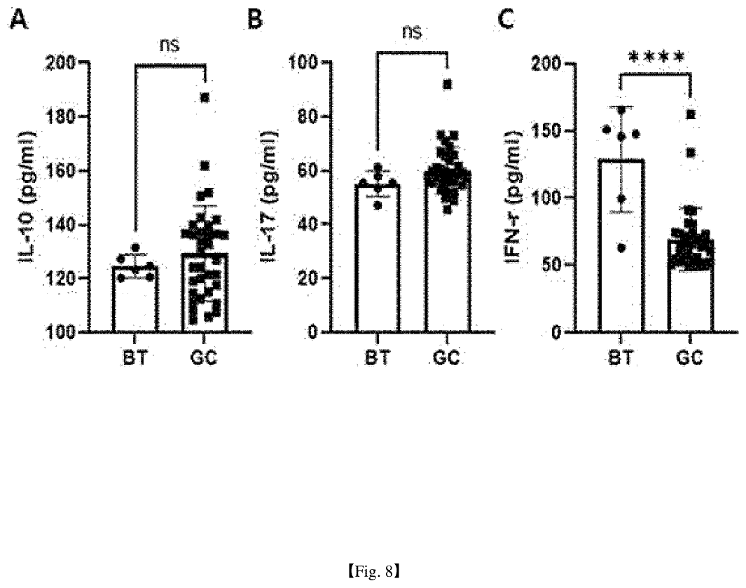

As a result, as shown in FIG. 8, it was confirmed that in the stomach cancer patient group (GC), compared to the benign tumor patient (BT), IL-10, a molecule related to immunity reduction was significantly increased, and IFN-γ was significantly decreased.

### <6-2> Identification of Immunity Reduction Markers of T Cells

In stomach cancer patients and benign tumor patients, to compare whether there was a difference in immunity of T cells, programmed cell death-1 (PD-1) expressed in T cells was confirmed when the immunity was reduced. Specifically, the expressions of PD-1 CD4+ cells and PD-1 CD8+ cells were confirmed by flow cytometry.

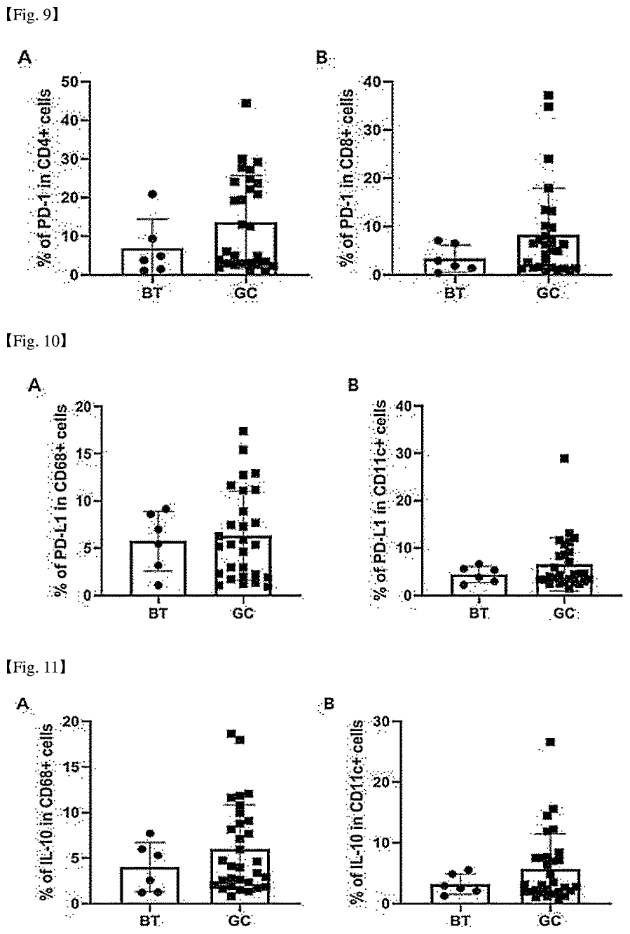

As a result, as shown in FIG. 9, it was confirmed that in the stomach cancer patient group (GC), compared to the benign tumor patient (BT), the expressions of PD-1 CD4+ cells and PD-1 CD8+ cells were significantly increased.

### <6-3> Identification of Immunity Reduction Markers in Antigen Presenting Cells

To compare the immune status of stomach cancer patients and benign tumor patients, in an antigen presenting cell (APC), the expression of Programmed Death-Ligand 1 (PD-L1) expressed by immunity reduction was confirmed. Specifically, in blood isolated from the patients, the expression of PD-L1, an immunosuppressive protein, in macrophages and dendritic cells was confirmed by immunohistochemical staining. In addition, the expression of IL-10 associated with immunity reduction was confirmed in the same manner as described above.

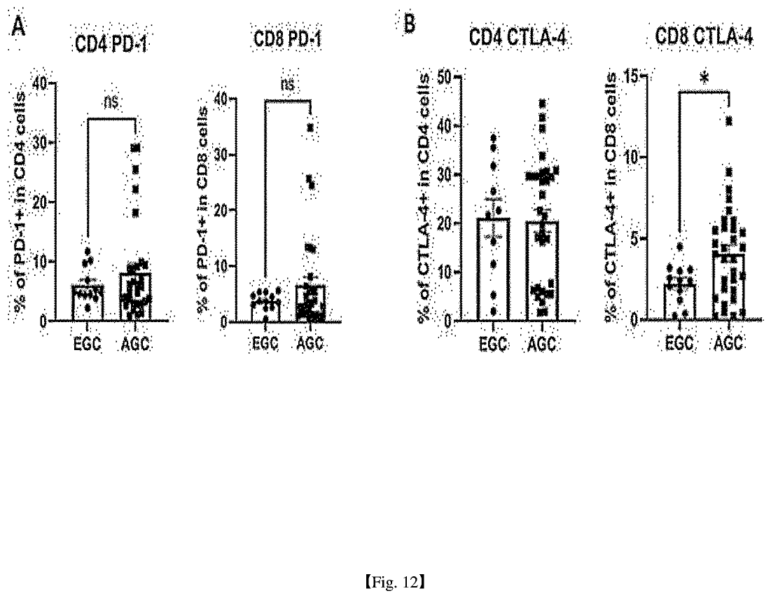

As a result, as shown in FIG. 10, it was confirmed that in the stomach cancer patient group (GC), compared to the benign tumor patient (BT), the expression of PD-L1 in APC was increased.

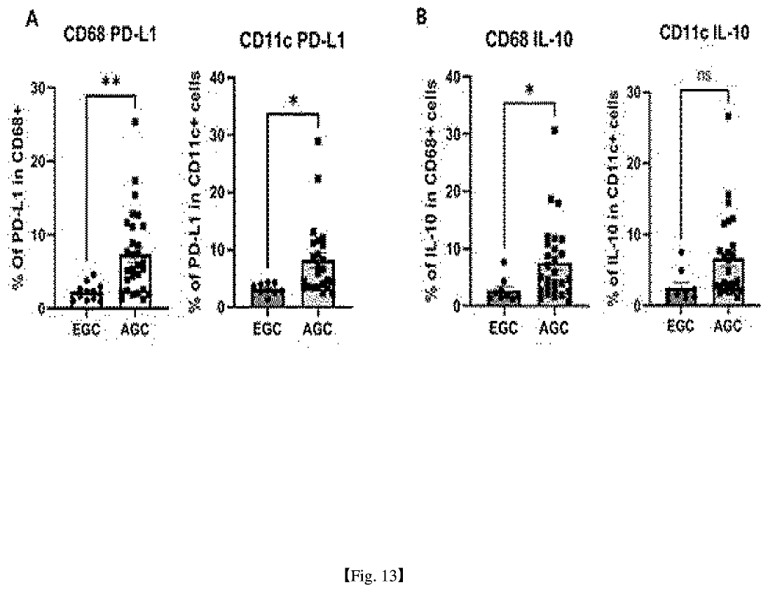

In addition, as shown in FIG. 11, it was confirmed that in the stomach cancer patient group (GC), compared to the benign tumor patient (BT), the expression of IL-10 in APC was also increased, and based on the result, it was confirmed that in the stomach cancer patient group, the APC suppressed the immune activity of T cells.

### <Example 7> Comparison of Immunity According to Stomach Cancer Progression

### <7-1> Confirmation of Immunity Reduction of T Cells

It was determined whether immunity was reduced in stomach cancer patients according to the progression of stomach cancer. Specifically, in the same manner as in Example 6-2, in T cells of early stomach cancer patients (EGC/stage 0 to 1a) and advanced stomach cancer patients (after AGC/stage 1b), the expression of PD-1 and CTLA-4, an immunity decline marker, was confirmed.

As a result, as shown in FIG. 12, it was confirmed that compared to the early stomach cancer patients (EGC), in the advanced stomach cancer patients (AGC), in T cells, the expression of PD-1 and CTLA-4, which were immunity reduction markers, was significantly increased.

### <7-2> Identification of Immunity Reduction Markers in Antigen Presenting Cells

To determine whether immunity was reduced in stomach cancer patients depending on the progression of stomach cancer, in the same manner as Example 6-3, in an early stomach cancer patient (EGC/stage 0 to 1a) and an advanced stomach cancer patient (after AGC/stage 1b), the expression of PD-L1 and IL-10 in APC was compared.

As a result, as shown in FIG. 13, it was confirmed that compared to the early stomach cancer patients (EGC), in the advanced stomach cancer patients (AGC), the expression of PD-L1 and IL-10, which were immunity reduction markers of APC, was increased.

### <7-3> Identification of Immunity Reduction Markers in Antigen Presenting Cells According to Progression of Stomach Cancer

As the progression of stomach cancer increased in stomach cancer patients, it was determined whether the expression of immunity reduction markers in antigen-presenting cells increased. In the same manner as Example 7-2, the blood was collected from a benign tumor patient (BT), a stage 1 stomach cancer patient (stage 1, I), a stage 2 stomach cancer patient (stage 2, II), a stage 3 stomach cancer patient (stage 3, III) and a stage 4 stomach cancer patient (stage 4, IV), and the expression of PD-L1 in macrophages and dendritic cells was intended to be confirmed from the blood.

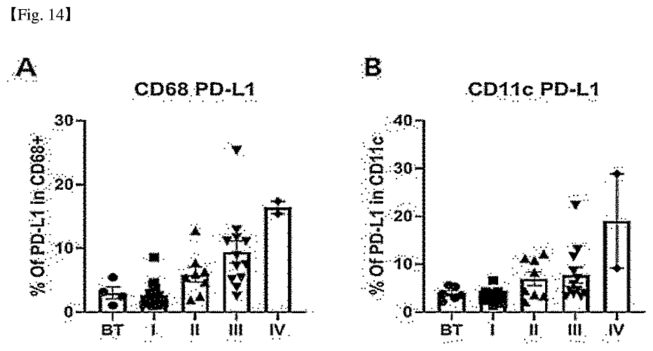

As a result, as shown in FIG. 14, it was confirmed compared to the benign tumor patient (BT), in the stomach cancer patient group, the expression of PD-L1 in macrophages and dendritic cells was all increased, and PD-L1 tended to be also increased as the progression stage of stomach cancer increased.

### <7-4> Confirmation of Expression of Immunity Reduction Markers in Stomach Cancer Tissue According to Progression of Stomach Cancer

To determine whether immunity was reduced in stomach cancer patients according to the progression of stomach cancer, the expression of immunity reduction markers in stomach cancer tissue were identified using a confocal microscope. Specifically, the expression of PD-L1 and IL-10 was compared in stomach cancer tissues of an early stomach cancer patient (EGC/stage 0 to 1a) and an advanced stomach cancer patient (after AGC/stage 1b).

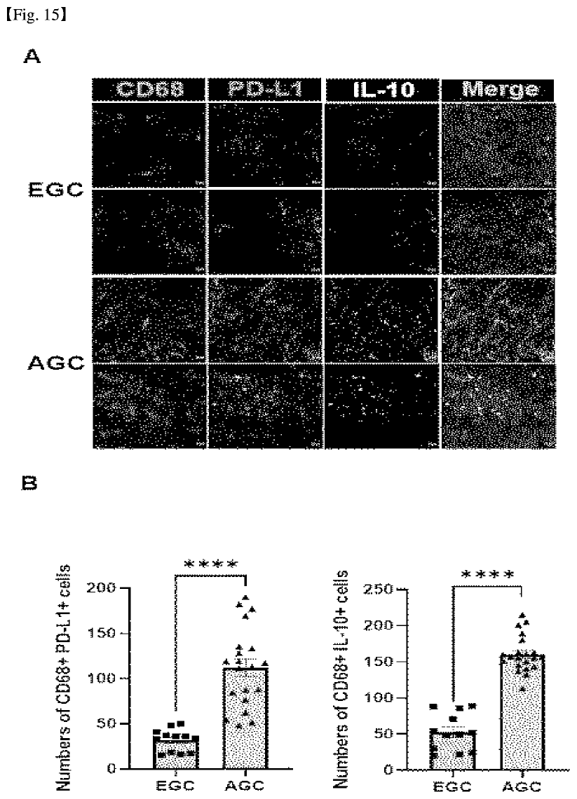

As a result, as shown in FIG. 15, it was confirmed that compared to the early stomach cancer patient (EGC), in the advanced stomach cancer patient (AGC), the expression of PD-L1 and IL-10, which were immunity reduction markers, was significantly increased in stomach cancer tissue.

### <Example 8> Confirmation of Increased Immunity According to Treatment with Sodium Butyrate and Faecalibacterium prausnitzii

Based on the results of Examples 5 to 7, it was intended to determine whether butyrate, which was a metabolite of Faecalibacterium, increased immunity. Specifically, in Example 6-3, peripheral mononuclear cells isolated from the patient were treated with butyrate at a concentration of 0.5 and 1 mM, and then incubated for 3 days, and thereafter, it was confirmed whether the expression of PD-L1 and IL-10 associated with immunity reduction was inhibited through flow cytometry. The macrophage marker was stained as a CD68 marker. As a control group, a Vehicle group in which the isolated peripheral mononuclear cells were treated with PBS was used. In addition, changes in expression of PD-L1 were further confirmed according to treatment with Faecalibacterium prausnitzii, a strain of the genus Faecalibacterium, which was shown to decrease in the stomach cancer patient group. In addition, when comparing a Nil group as an untreated control group, a Vehicle group treated with PBS, and a group treated with butyrate in the peripheral mononuclear cells isolated from the patient, the activity of macrophages expressing PD-L1 and IL-10 was analyzed, and the expression of IL-10 in the culture medium was further confirmed, and the activity of macrophages expressing PD-L1 and IL-10 was analyzed according to treatment with Faecalibacterium prausnitzii.

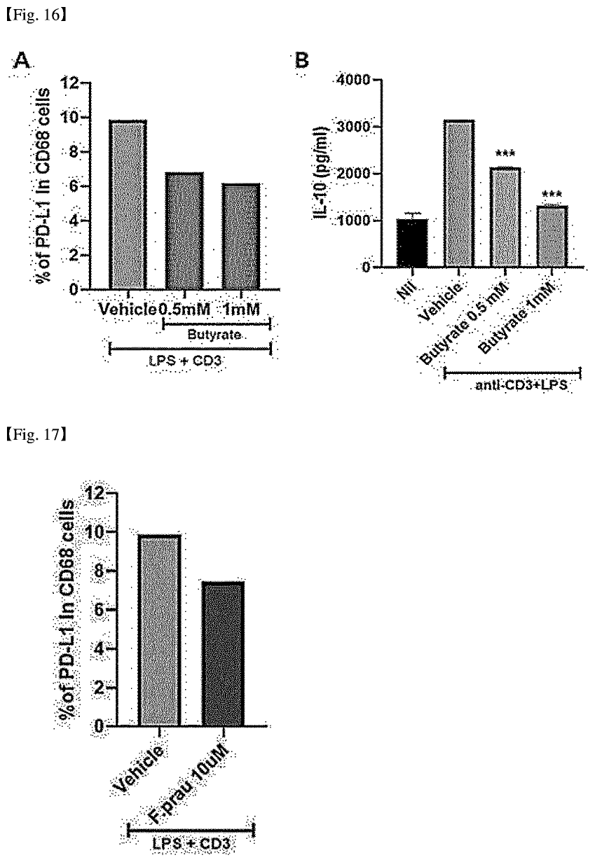

As a result, as shown in FIG. 16, it was confirmed that compared to the Vehicle group, in the group treated with butyrate, the expression of PD-L1 and IL-10 was decreased in a concentration-dependent manner in CD68 positive cells, a macrophage marker. In addition, it was confirmed that the expression of PD-L1 in macrophages was significantly reduced even by treatment with Faecalibacterium prausnitzii of the present invention (FIG. 17).

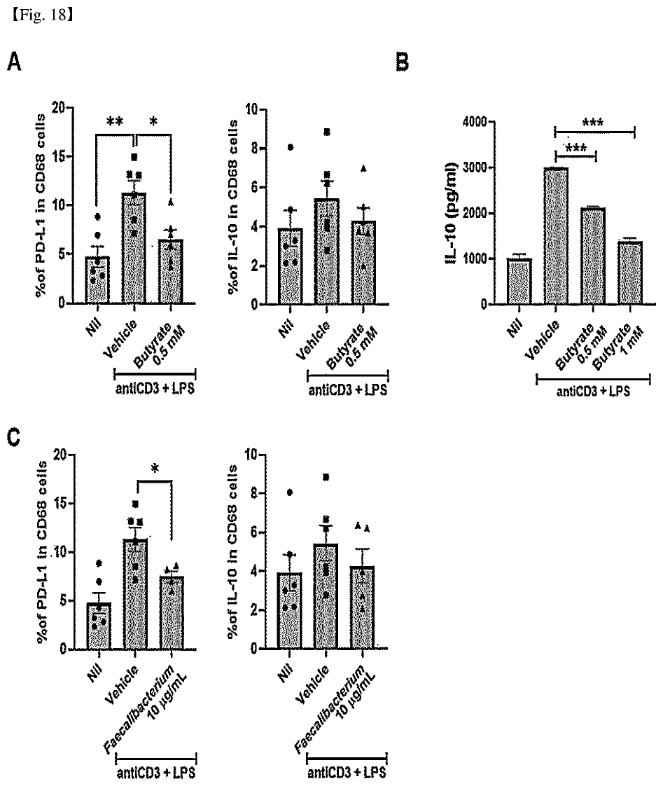

In addition, as shown in FIG. 18, it was confirmed that compared to the Nil group, in the Vehicle group, the expression of PD-L1 and IL-10, immunity reduction markers, in macrophages was increased and IL-10 in the culture medium increased significantly, but PD-L1 and IL-10 increased in the group treated with butyrate, and IL-10 in the culture medium were significantly decreased (FIGS. 18A and 18B). In addition, it was confirmed that the increased expression of PD-L1 and IL-10 was significantly reduced by treatment with Faecalibacterium prausnitzii (FIG. 18C), and it was confirmed that treatment with butyrate or Faecalibacterium prausnitzii of the present invention increased the immunity of macrophages in the stomach cancer patient.

### <Example 9> Confirmation of Killing Effect of Faecalibacterium prausnitziiand Metabolites on Stomach Cancer Cells

<9-1> Confirmation of Cell Killing Effect of Faecalibacterium prausnitzii

In the present invention, it was intended to determine whether Faecalibacterium prausnitzii, which was found to be reduced in stomach cancer patients, induced apoptosis in a stomach cancer cell line. Specifically, AGS cells, a stomach cancer cell line, were incubated, the incubated AGS cells were treated with Faecalibacterium prausnitzii of the present invention at a concentration of 10 μg/ml, and the cells in which apoptosis was induced were analyzed by flow cytometry. As a control group, a Vehicle group treated with the same amount of PBS was used.

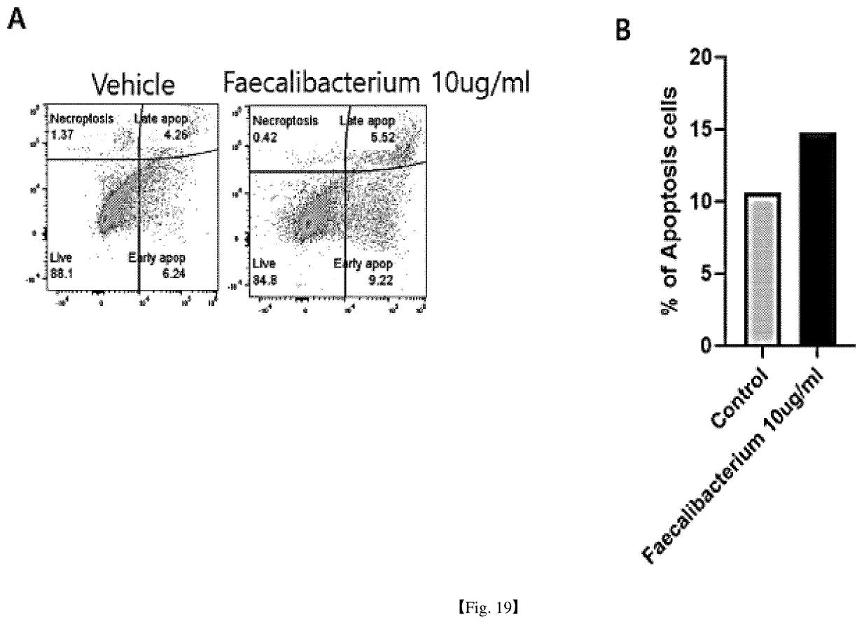

As a result, as shown in FIG. 19, it was confirmed that compared to the Vehicle group, apoptosis cells increased in the group treated with Faecalibacterium prausnitzii.

<9-2> Confirmation of Cell Killing Effect of Metabolite of Faecalibacterium prausnitzii

It was intended to confirm whether butyrate, a metabolite of Faecalibacterium prausnitzii of the present invention, induced apoptosis in the same manner as Faecalibacterium prausnitzii. Specifically, in the same manner as Example 9-1, the AGS cell line was treated with butyrate at concentrations of 0.5, 1, and 1.5 mM, and cells in which apoptosis was induced were analyzed by flow cytometry. In addition, in a stomach cancer cell line, the cell killing effect of butyrate was confirmed by measuring the cell viability and absorbance at a wavelength of 450 nm.

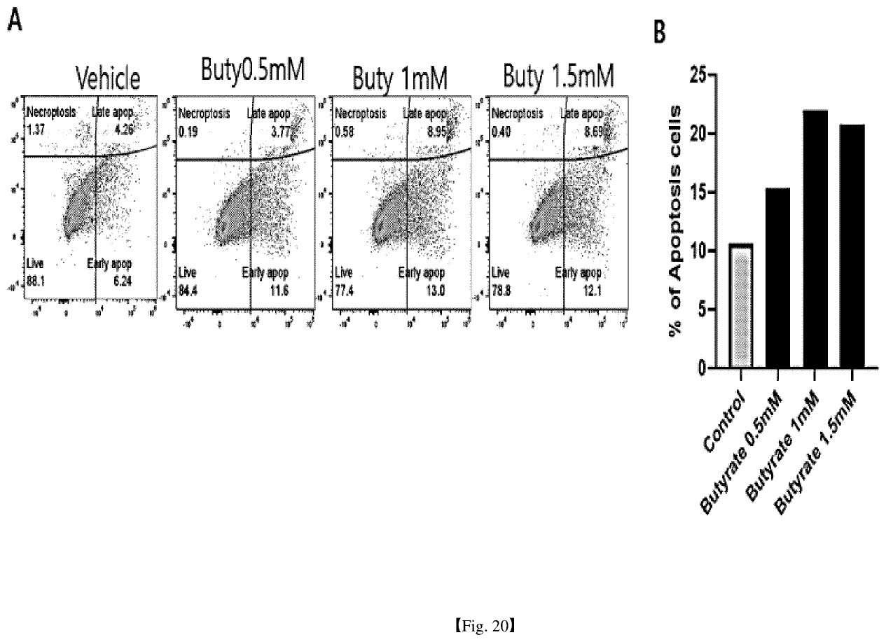

As a result, as shown in FIG. 20, it was confirmed that compared to the Vehicle group, apoptosis was induced in a concentration-dependent manner in the butyrate-treated group.

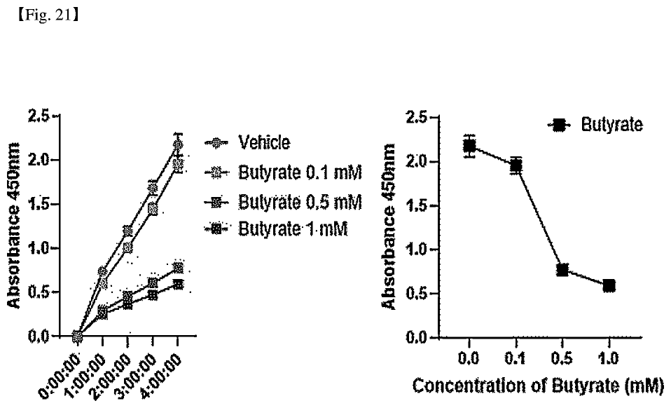

In addition, as shown in FIG. 21, it was confirmed that compared to the Vehicle group, in the butyrate-treated group, the absorbance was decreased in a concentration-dependent manner to induce the apoptosis of the stomach cancer cell line.

### <Example 10> Confirmation of Tumor Inhibition of Butyrate in Stomach Cancer Patient Simulation Avatar Model

### <10-1> Fabrication of Stomach Cancer Patient Simulation Avatar Model and Confirmation of Tumor Inhibition of Butyrate

In order to confirm whether butyrate, a metabolite of Faecalibacterium prausnitzii of the present invention, inhibited tumors, a stomach cancer patient simulation avatar model was fabricated. Specifically, 6˜8 week-old immunodeficient mice (NSG) were intravascularly injected with peripheral blood mononuclear cells (PBMCs) derived from stomach cancer patients at 5×10⁶/mice. One week after PBMC injection, 5×10⁶ of an AGS cell line, a stomach cancer cell line, was injected subcutaneously to fabricate a stomach cancer patient simulation avatar model (AGS+PBMC, Vehicle (PBMC)). In addition, in order to confirm the injection effect of PBMCs derived from stomach cancer patients on the engraftment of a stomach cancer cell line, a group administered with only AGS (AGS only) was used as a control group. Three weeks after AGS cell injection, butyrate was orally administered daily at a concentration of 200 mg/kg. After AGS cell injection, tumor size was measured three times weekly using calipers, and tumor volume was calculated by substituting the lengths in long axis and short axis of the tumor into Equation 1 above.

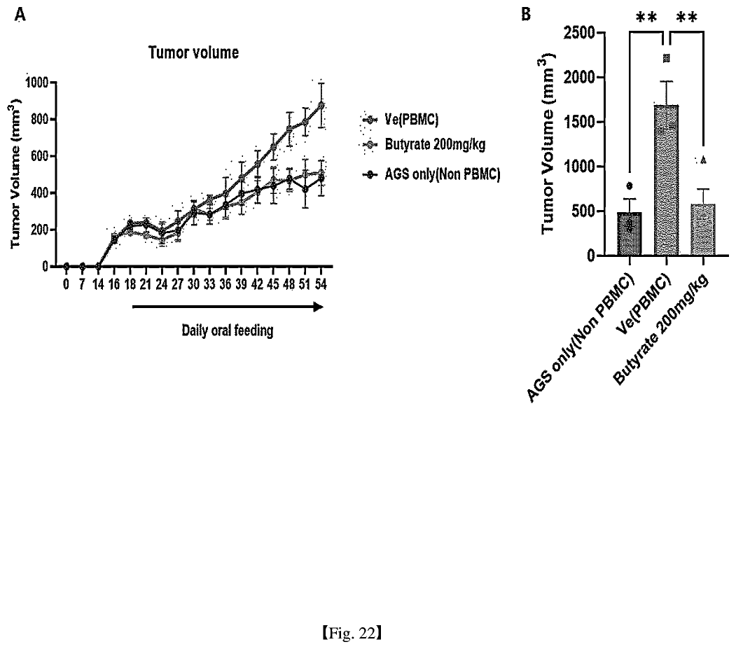

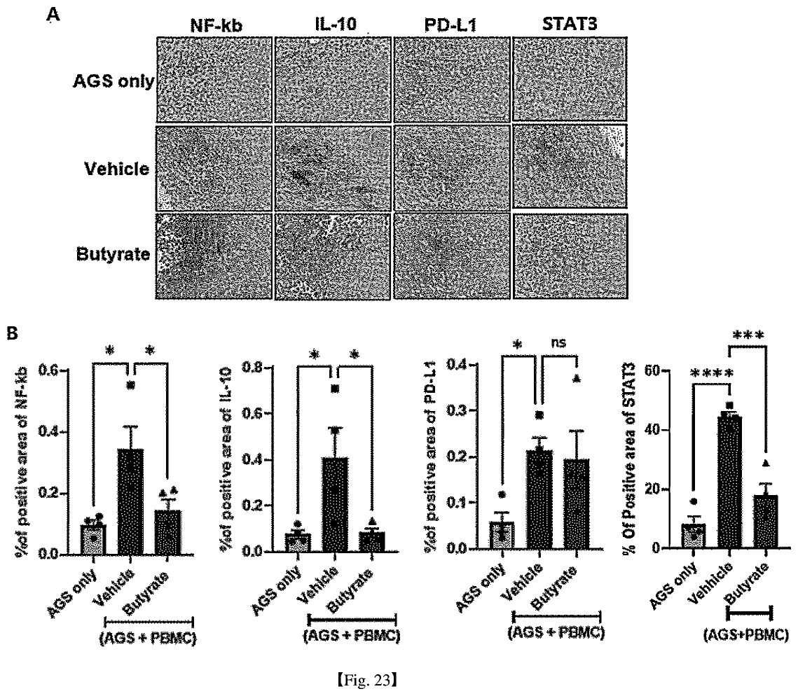

As a result, as shown in FIG. 22, it was confirmed that compared to the AGS only group, in the group injected with the patient-derived PBMCs, the tumor growth was significantly increased and the tumor volume was increased to reflect the immune status of the patient, and in the group treated with butyrate, the increased tumor volume was significantly decreased to confirm the tumor inhibitory effect of butyrate.

<10-2> Confirmation of Expression of Tumor Markers and Immunity reduction markers in stomach cancer patient simulation avatar model

It was confirmed whether butyrate, a metabolite of Faecalibacterium prausnitzii of the present invention, regulated the expression of tumor markers and immunity reduction markers. Specifically, mice of each group in Example 10-1 were humanely sacrificed, and then transplanted tumor tissues were obtained. Thereafter, the obtained tissue was segmented, and then the expression of tumor markers NF-κb and STAT3 and immunity reduction markers PD-L1 and IL-10 was confirmed by immunohistochemical staining.

As a result, as shown in FIG. 23, it was confirmed that compared to the AGS only group, in the Vehicle group, the expression of tumor markers and immunity reduction markers NF-κb, STAT3, PD-L1, and IL-10 was significantly increased, but in the butyrate-treated group, the increased tumor markers and immunity reduction markers were significantly decreased, so that butyrate had tumor inhibitory and immunomodulatory effects even in an avatar model in which the immune status of patients was reflected.

Accordingly, it has been identified that a novel compound SD217 of the present invention specifically kills stomach cancer cells and effectively inhibits the expression of PD-L1, which is a surface protein expressed by stomach cancer cells in order to evade immune responses of the human body, and thus kills stomach cancer cells. In addition, it has been identified that tumor size is suppressed in a stomach cancer tumor animal model. In addition, it has been identified that intestinal microflora diversity decreases in stomach cancer patients and the composition ratio of the intestinal microflora differs from those of healthy adults and benign tumor patients. In addition, it has been identified that the immune function declines in stomach cancer patient groups and the immune function of patients declines as the stomach cancer progresses. In addition, it has been identified that PD-L1 and IL-10, which increase because of reduced immunity, are effectively inhibited when macrophages derived from patients are treated with a Faecalibacterium prausnitzii strain, which has been identified to be reduced in stomach cancer patient groups, and butyrate, which is a metabolite thereof. In addition, it has been identified that cell killing is effectively induced when a stomach cancer cell line is treated with a Faecalibacterium prausnitzii strain and butyrate, which is a metabolite thereof. In addition, it has been identified that even in a stomach cancer avatar animal model in which the immune status of patients is reflected and into which a stomach cancer cell line is transplanted, butyrate inhibits tumor growth and reduces the expression of tumor markers and immunity reduction markers. Therefore, intestinal microflora dysbiosis and reduced immunity in stomach cancer patient groups are diagnosed such that the effects of increased immunity and intestinal microflora recovery according to the treatment of an active ingredient of the present invention have been identified, and thus it has been identified that immune anticancer drugs and individual personalized medicine may be implemented.

1. A method for preventing or treating of cancer comprising administering a pharmaceutical composition to a subject in need thereof comprising a compound represented by Chemical Formula 1 below or a pharmaceutically acceptable salt thereof as an active ingredient, 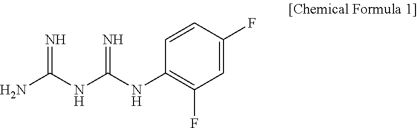 [SMILES: N=C(N)NC(=N)Nc1ccc(F)cc1F]
wherein the composition inhibits the growth of cancer cells.

2. The method of claim 1, wherein the composition increases apoptosis of cancer cells,
wherein the apoptosis is early or late apoptosis.

3. The method of claim 1, wherein the composition inhibits the expression of Programmed Death-Ligand 1 (PD-L1), which is a surface protein of cancer cells.

4. The method of claim 1, wherein the cancer is cancer selected from the group consisting of stomach cancer, colon cancer, rectal cancer, anal cancer, bone cancer, cerebrospinal tumor, head and neck cancer, thymoma, mesothelioma, esophageal cancer, biliary tract cancer, bladder cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer.

5. An anticancer adjuvant comprising a compound represented by Chemical Formula 1 below or a pharmaceutically acceptable salt thereof as an active ingredient.

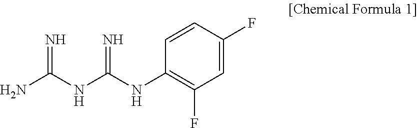 [SMILES: N=C(N)NC(=N)Nc1ccc(F)cc1F]

6. A method for preventing or treating of cancer comprising administering a compound represented by Chemical Formula 1 below or a pharmaceutically acceptable salt thereof as an active ingredient in a pharmaceutically effective amount to a subject with improved expression of Programmed Death-Ligand 1 (PD-L1)

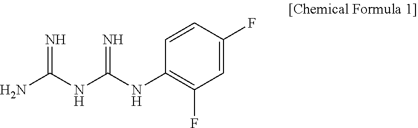 [SMILES: N=C(N)NC(=N)Nc1ccc(F)cc1F]

7. The method of claim 6, wherein the composition decreases the expression of PD-L1.

8. The method of claim 7, wherein the decreasing of the expression of PD-L1 increases the cancer-specific activity of immune cells.

9. A method for preventing or treating of cancer comprising administering a pharmaceutical composition comprising butyrate or a microbiome as an active ingredient,
wherein the microbiome is Faecalibacterium prausnitzii,
wherein the composition increases an immune function.

10. The method of claim 9, wherein the increasing of the immune function inhibits the expression of IL-10, IL-17, and INF-γ, which are immunity reduction factors.

11. The method of claim 10, wherein the increasing of the immune function inhibits the expression of programmed cell death-1 (PD-1) or CTLA-4 of T cells; and
wherein the increasing of the immune function inhibits the expression of Programmed Death-Ligand 1 (PD-L1) and IL-10 in antigen presenting cells (APC) or cancer tissue.

12. The method of claim 11, wherein the antigen presenting cells are macrophages or dendritic cells.

13. The method of claim 9, wherein the composition increases apoptosis of cancer cells.

14. The method of claim 9, wherein the cancer is selected from the group consisting of stomach cancer, colon cancer, rectal cancer, anal cancer, bone cancer, cerebrospinal tumor, head and neck cancer, thymoma, mesothelioma, esophageal cancer, biliary tract cancer, bladder cancer, testicular cancer, small intestine cancer, seminoma, endometrial cancer, fallopian tube carcinoma, vaginal carcinoma, vulvar carcinoma, multiple myeloma, sarcoma, endocrine cancer, thyroid cancer, parathyroid cancer, adrenal cancer, bladder cancer, urethral cancer, pituitary adenoma, renal pelvic carcinoma, spinal cord tumor, multiple myeloma, glioma cancer, central nervous system (CNS) tumor, hematopoietic tumor, fibrosarcoma, neuroblastoma, astrocytoma, breast cancer, cervical cancer, ovarian cancer, prostate cancer, pancreatic cancer, kidney cancer, liver cancer, brain cancer, lung cancer, lymphoma, leukemia, malignant melanoma, and skin cancer.

15. A method for increasing Faecalibacterium sp. strains and decreasing Proteobacteria Phylum strains in cancer patient groups, comprising administering a composition including butyrate or a microbiome as an active ingredient to a subject.

16. A method for increasing a T cell function and an antigen presenting cell (APC) function in cancer patient groups, comprising administering a composition including butyrate or a microbiome as an active ingredient to a subject.

17. The method of claim 16, wherein the increasing of the T cell function inhibits the expression of programmed cell death-1 (PD-1) or CTLA-4 of T cells; and wherein the increasing of the immune function inhibits the expression of Programmed Death-Ligand 1 (PD-L1) and IL-10 in antigen presenting cells (APC) or cancer tissue.

18. The method of claim 17, wherein the increasing of the T cell function inhibits the expression of Programmed Death-Ligand 1 (PD-L1) and IL-10 of the antigen presenting cells.
